(19) United States
(12) Patent Application Publication
(10) Pub. No.: US 2026/0294273 A1
(43) Pub. Date: Oct. 1, 2026

(54) SLEEP-RELATED BREATHING DISORDER MONITORING METHOD AND SYSTEM, AND ELECTRONIC DEVICE

(71) Applicant: University of Science and Technology of China, Hefei (CN)

(72) Inventors: Tingrui PAN, Suzhou (CN); Kairui WU, Suzhou (CN); Liang YANG, Suzhou (CN); Ruojiang WANG, Suzhou (CN); Binyang DING, Suzhou (CN)

(73) Assignee: University of Science and Technology of China, Hefei (CN)

(21) Appl. No.: 19/332,087

(22) Filed: Sep. 18, 2025

(30) Foreign Application Priority Data
Mar. 27, 2025 (CN) 202510372657.9

(51) Int. Cl. A61B 5/08; A61B 5/00; A61B 5/256; A61B 5/369; A61B 5/398

(52) U.S. Cl. CPC A61B 5/0826; A61B 5/256; A61B 5/369; A61B 5/398; A61B 5/4812

(57) ABSTRACT

A sleep-related breathing disorder monitoring method includes: acquiring an electroencephalogram (EEG) signal, an electrooculogram (EOG) signal, and a photoplethysmography (PPG) signal; performing automatic sleep staging and sleep time (ST) calculation based on the EEG signal and the EOG signal; counting apnea and hypopnea events based on the PPG signal; and calculating an apnea-hypopnea index (AHI) based on a calculated sleep time (ST) and the number of apnea and hypopnea events. Furthermore, a corresponding head-worn sleep-related breathing disorder monitoring system and electronic device is provided. The sleep-related breathing disorder monitoring method can achieve real-time and accurate sleep staging and apnea diagnosis.

### CROSS REFERENCE TO THE RELATED APPLICATIONS

This application is based upon and claims priority to Chinese Patent Application No. 202510372657.9, filed on Mar. 27, 2025, the entire contents of which are incorporated herein by reference.

### TECHNICAL FIELD

The present invention belongs to the technical field of sleep-related breathing disorder monitoring, and in particular relates to a head-worn sleep-related breathing disorder monitoring technique based on multi-modal signals.

### BACKGROUND

Sleep staging divides the sleep into rapid eye movement (REM) sleep and non-rapid eye movement (NREM) sleep based on physiological signals during the night. The REM sleep is a stage when dreaming is most likely to occur. During this stage, muscle activity reaches its lowest level throughout the night, and rapid eye movements can be observed. The NREM sleep includes three stages: light sleep stage (N1), moderate sleep stage (N2), and deep sleep stage (N3). In the light sleep stage, the human body is easily awakened. The moderate sleep stage accounts for the largest proportion of total sleep time (ST), signifying the human body entering deeper sleep. The deep sleep stage is also known as the slow-wave sleep stage, which is primarily responsible for human body recovery and growth. In a typical sleep cycle of healthy adults, the human body generally first transitions from wakefulness to the N1 stage of the NREM sleep, during which attention gradually diminishes and slow eye movements occur. After several minutes, the human body enters the N2 stage where muscle and eye movements decrease. When the human body enters the N3 stage, the muscle tone further declines, making the human body difficult to awaken. When the N3 stage ends, the human body sequentially experiences the N2 and N1 stages before transitioning to the REM sleep. When the REM sleep ends, the human body enters another N1 stage, and the sleep cycle repeats.

Obstructive sleep apnea (OSA) is one of the common sleep disorders, characterized by repeated upper airway obstruction during sleep, typically causing a decrease in the blood oxygen saturation. Generally, patients are unaware of their sleep apnea and require detection and reminders from their bed partners, posing significant hidden risks. Currently, the simplest and most direct clinical method to terminate apnea is to awaken the patient. Apnea ceases upon awakening, and the blood oxygen saturation gradually returns to a baseline level after normal breathing resumes. An apnea event refers to the complete cessation of oral and nasal airflow for 10 seconds or more during sleep. A hypopnea event refers to a reduction in respiratory airflow intensity (amplitude) by 50% or more from the baseline level, accompanied by a drop of 4% or more in the blood oxygen saturation from the baseline level. The apnea-hypopnea index (AHI) refers to the total number of apnea and hypopnea events per hour during sleep.

Polysomnography (PSG) is a multi-channel physiological monitoring technique presented to clinicians after filtering, amplification, and other processing of bioelectrical signals from different body parts (such as the brain, eyes, jaw) and physiological signals like nasal airflow and snoring acquired by PSG devices. PSG is acquired and recorded by professional physicians or technicians in hospitals or sleep centers for diagnosing various sleep disorders. PSG is the gold standard for diagnosing sleep apnea-hypopnea syndrome. Therefore, in current clinical diagnosis, the calculation of AHI typically requires monitoring overnight sleep-related breathing disorders using PSG devices. However, PSG devices have the following significant shortcomings in portability and intelligence. (1) PSG devices are expensive and bulky, limiting their use to hospitals, sleep research centers, and similar settings. (2) The use of PSG devices requires assistance from professional technicians, and others are incompetent for independently installing and operating PSG devices. (3) Data acquisition typically requires at least 12 channels, necessitating at least 22 data cables connected between the subject and the data acquisition device, causing subject discomfort and affecting test accuracy. (4) Overnight sleep data acquisition requires professional processing and analysis, consuming significant effort and lacking real-time capability. Furthermore, although PSG serves as the gold standard for monitoring sleep-related breathing disorders and diagnosing various sleep disorders, it exhibits redundancy in the types of signals required for sleep-related breathing disorders.

OSA can occur during any stage of sleep. OSA is more likely to occur during N1, N2, and REM stages compared to the N3 stage. When OSA occurs during the REM stage, the duration of apnea is longer, and the blood oxygen saturation decreases more rapidly. Therefore, diagnosing sleep-related breathing disorders requires not only identifying apneas and hypopneas but also accurately determining the sleep stages. According to the American Academy of Sleep Medicine (AASM) Manual for the Scoring of Sleep and Associated Events: Rules, Terminology and Technical Specifications, sleep staging primarily relies on electroencephalogram (EEG) and electrooculogram (EOG) signals. Since rapid eye movements occur during the REM stage, the REM stage can be accurately identified through EOG signals. However, EOG electrodes, with complex placement and positioning, are located on both sides of the face and are prone to compression during sleep in a lateral position. Consequently, currently, there are few head-worn devices for detecting EOG signals. EEG signals are weak voltage signals at the microvolt (μV) level, with their amplitude and waveform susceptible to the positions of acquisition electrodes and reference electrodes. Due to different positions of the acquisition electrodes and the reference electrodes, existing monitoring devices suffer from non-standardization issues. As a result, most acquired signals fail to meet the medical sleep staging scoring standard in amplitude and waveform, rendering them uninterpretable by professional physicians. Therefore, the accuracy of sleep staging algorithms developed on the basis of unknown signal quality cannot be determined. Additionally, current sleep staging algorithms mostly rely on overnight data analysis, making real-time sleep staging difficult to achieve.

For example, Chinese patent application CN116035557A discloses a flexible wearable sleep apnea monitoring system. The disclosure acquires electrocardiogram (ECG), blood oxygen saturation, body position changes, and other information from the chest and calculates AHI based on the acquired information. However, it relies solely on body position changes to calculate the ST. The calculation of AHI requires sleep staging to obtain precise ST. According to the AASM Manual for the Scoring of Sleep and Associated Events: Rules, Terminology and Technical Specifications, sleep staging primarily relies on EEG and EOG signals. Therefore, relying solely on body movements to calculate ST can easily lead to errors in AHI calculation. Meanwhile, the disclosure employs a machine learning method to calculate AHI, heavily relying on manually selected features, resulting in poor algorithm stability and generalizability. For another example, Chinese patent application CN119344671A discloses a portable EEG-blood oxygen saturation-ECG-based sleep-related breathing disorder monitoring device. The disclosure acquires EEG and blood oxygen saturation signals from the frontal region via surface electrodes and ECG signals from the chest via patch electrodes. In this disclosure, EEG electrodes are all attached to the forehead, there are no EOG electrodes, and the reference electrodes are not placed according to the international standard (international 10-20 system). Consequently, the disclosure cannot eliminate artifacts from eye movements and struggles to acquire accurate EEG signals with clear amplitude and waveform.

Developed by Dreem, Dreem2 is a wireless headband for monitoring sleep-related breathing disorders. It can help users understand and improve their sleep quality by monitoring their EEG signals, heart rate (HR), and body movement data. Dreem2 is currently one of the most accurate consumer-grade sleep trackers on the market and has received approval from the Food and Drug Administration (FDA) for sale as a Class II medical device. Dreem2 has six EEG electrodes: four located on the forehead (prefrontal region) and two on the back of the head (occipital region). When the device operates, one electrode serves as the reference electrode and does not participate in potential measurement. The frontal portion of the headband includes a pulse oximeter for measuring HR. However, the EEG electrodes of Dreem2 are closely spaced, with all the four frontal electrodes positioned over the frontal region. Consequently, the potential differences in the acquired signals are small, and signals acquired by the EEG electrodes are similar, resulting in considerable redundant information. Eye movements can cause changes in electrical signals that can spread across the entire scalp, with amplitudes reaching up to 100 mV. In contrast, EEG signals are weak, typically with amplitudes around 50 μV. Therefore, EEG signals are susceptible to artifacts from eye movements and require combination with EOG signals to eliminate the artifacts. The reference electrode of the Dreem2 headband is located either in the frontal region or the occipital region. However, if the reference electrode is located in the frontal region, it is too close to the EEG electrodes, thereby leading to high signal similarity and small potential differences, making it hard to derive valid EEG signals. During sleep, the electrode located in the occipital region is prone to displacement due to head movements, leading to inaccurate and unstable reference potentials.

In summary, PSG devices suffer from issues such as complex setup, non-wearability, and poor comfort. Existing commercial wearable sleep-related breathing disorder monitoring devices cannot simultaneously achieve sleep stage determination, sleep-related breathing disorder monitoring, and accurate AHI calculation. Furthermore, the existing head-worn sleep-related breathing disorder monitoring device Dreem2 relies solely on EEG signals, is susceptible to artifacts from eye movements, and is unreasonable in the setup of the reference electrode.

### SUMMARY

To solve the above technical problems, the present invention provides a sleep-related breathing disorder monitoring method for a head-worn sleep-related breathing disorder monitoring system. The present invention optimizes electrode selection and placement while meeting sleep diagnosis requirements, solving the problems of EEG signals being interfered by eye movements and excessive common-mode interference caused by insufficient inter-electrode spacing. The present invention enables simultaneous multi-channel data analysis of EEG, EOG, and blood oxygen saturation signals, achieving real-time and accurate sleep staging and apnea diagnosis. Furthermore, the present invention provides a corresponding head-worn sleep-related breathing disorder monitoring system and electronic device.

The present invention adopts the following technical solutions.

A first aspect of the present invention provides a sleep-related breathing disorder monitoring method, applied to a head-worn sleep-related breathing disorder monitoring system, and including:
acquiring an electroencephalogram (EEG) signal, an electrooculogram (EOG) signal, and a photoplethysmography (PPG) signal;
performing automatic sleep staging and sleep time (ST) calculation based on the EEG signal and the EOG signal;
counting apnea and hypopnea events based on a blood oxygen saturation calculated from the PPG signal; and
calculating an apnea-hypopnea index (AHI) based on a calculated sleep time (ST) and the number of apnea and hypopnea events.

A second aspect of the present invention provides a head-worn sleep-related breathing disorder monitoring system, including: a head-worn sleep-related breathing disorder monitoring device and a computer program, where the head-worn sleep-related breathing disorder monitoring device includes a head-worn wearable mechanism, and EOG electrodes, EEG electrodes, reference electrodes, a PPG sensor and a hardware circuit that are disposed on the head-worn wearable mechanism; there are a pair of EOG electrodes, a pair of EEG electrodes, and a pair of reference electrodes; when the head-worn wearable mechanism is worn, the pair of EOG electrodes are respectively positioned below an outer canthus of a left eye and above an outer canthus of a right eye of a human body to acquire an EOG signal; the pair of EEG electrodes are respectively positioned at left and right sides of a prefrontal region of the human body; the pair of reference electrodes are respectively positioned to contact mastoid processes posterior to junctions of left and right auricles with cheeks of the human body; the EEG electrodes and the reference electrodes cooperate to acquire an EEG signal; the PPG sensor is configured to acquire a PPG signal of the human body; the hardware circuit is configured to preprocess the EOG signal, the EEG signal, and the PPG signal; one or more computer programs are stored in the hardware circuit and/or an electronic device communicatively connected to the head-worn sleep-related breathing disorder monitoring device; and the computer program is executed to implement the sleep-related breathing disorder monitoring method according to the first aspect and any optional solution of the present invention.

A third aspect of the present invention provides an electronic device, including: a processor, a memory, and a second communication module, where the electronic device is communicatively connected to head-worn sleep-related breathing disorder monitoring device through the second communication module; and the processor is configured to call a computer program stored in the memory to implement the sleep-related breathing disorder monitoring method according to the first aspect of the present invention.

The present invention has the following beneficial effects:
(1) In traditional methods, the AHI is calculated based on acquired overnight ST, ignoring errors caused by intermediate awakenings, which can easily lead to underestimated AHI values. The integrated head-worn device of the present invention can detect and count both apnea and hypopnea events while monitoring sleep state, further improving the accuracy of AHI calculation.
(2) In the present invention, both EEG and EOG signals acquired by the head-worn sleep-related breathing disorder monitoring device serve as the basis for sleep staging. The EEG signal provides a characteristic brain wave to accurately distinguish different sleep stages (N1, N2, and N3), while the EOG signal reflects eye movements to aid in identifying the REM stage. These two signals are combined to eliminate signal interference from ocular artifacts. Compared to a single EEG or EOG signal, the combined signal can provide more comprehensive information, enabling more accurate sleep staging.
(3) The head-worn sleep-related breathing disorder monitoring device provided by the present invention is an integrated head-worn wearable device. Through optimized electrode type selection and placement design, the present invention solves the problems of EEG signals being interfered by eye movements and excessive common-mode interference caused by insufficient inter-electrode spacing. Additionally, the present invention can simultaneously acquire three signals, namely EEG, EOG, and blood oxygen saturation, and perform synchronized accurate sleep staging and sleep-related breathing disorder monitoring, achieving real-time and accurate AHI calculation.

In particular, the present invention optimizes the placement of reference electrodes. The reference electrodes are arranged on the inner sides of the straps of the eye mask and located posterior to junctions of auricles with cheeks (overlying the mastoid processes) to provide reference potential signals when the eye mask is worn. The potential signals acquired by the left and right EEG electrodes located on the forehead and the potential differences of the reference signals acquired by the left and right reference electrodes form two EEG signals. Meanwhile, the reference electrode placement site features thin skin and thick bone, and is distant from the cerebral cortex, making it less susceptible to interference from factors such as sweat, body movement, and brain activity. Therefore, the reference electrodes can provide stable reference signals.

Furthermore, the present invention optimizes the placement of the EEG and EOG electrodes located on the head and face. The present invention optimizes the electrode placement based on the forehead length and width, intercanthal distance, and eye width of Asian adults. Therefore, the electrode placement complies with international PSG wearing standards to ensure high consistency and interpretability of signal characteristics compared to clinical-grade devices.
(4) In the present invention, the loop side and hook side of the flexible hook-and-loop fastener work together to achieve flexible adjustment and precise positioning of electrode placement, accommodating deviations caused by differences in human facial structures. During initial wear, users can accurately adjust electrode positions according to their facial structure characteristics, avoiding readjustments during subsequent wear.
(5) The present invention improves the convolutional neural network (CNN) in the sleep staging algorithm by combining the CNN with long short-term memory (LSTM) to effectively extract signal features and temporal sequence relationships. In the CNN, multiple different convolutional layers extract local features of EEG and EOG signals from different dimensions, effectively capturing rhythmic changes of EEG signals and eye movement signals. In the LSTM, feature sequences replace original signals, reducing computational complexity, achieving lightweight modeling, and facilitating rapid and sensitive perception of temporal changes in sleep stages by the LSTM.
(6) The present invention can calculate the blood oxygen saturation in real time based on photoplethysmography (PPG) signals acquired by the head-worn sleep-related breathing disorder monitoring device and monitor changes in the oxygen saturation in real time. The present invention can accurately capture OSA and hypopnea events by identifying nocturnal hypoxemia events and cyclic desaturation trends. Compared to traditional respiratory airflow monitoring, the blood oxygen saturation monitoring sensor in the present invention is smaller, offers higher wearing comfort, and has stronger system integration.
(7) The head-worn sleep-related breathing disorder monitoring device provided by the present invention uses a flexible fabric and wireless data transmission, offering strong wearing comfort without requiring data cable connections to transmit data. Meanwhile, the present invention uses flexible and stretchable electrodes and wires to meet the deformation and extension requirements of the eye mask, thereby establishing a long-term, stable skin-electrode contact interface to achieve accurate data acquisition over extended periods during the night.

### BRIEF DESCRIPTION OF THE DRAWINGS

FIG. 1 is a schematic structural diagram 1 of a sleep-related breathing disorder monitoring eye mask;

FIG. 2 is a schematic structural diagram 2 of the sleep-related breathing disorder monitoring eye mask;

FIG. 3 is a front perspective view of the sleep monitoring eye mask in a worn state;

FIG. 4 is a left-side perspective view of the sleep monitoring eye mask in the worn state;

FIGS. 5A-5B are schematic structural diagrams of an electrode structure and a connecting wire of the sleep monitoring eye mask;

FIG. 6 is a schematic diagram of an electrode bonded to a hook-and-loop fastener;

FIG. 7 is a block diagram of circuit connection of a sleep-related breathing disorder monitoring system;

FIG. 8 is a flowchart of AHI calculation;

FIG. 9 is a schematic diagram of a network structure of a sleep staging module;

FIG. 10 is a structural diagram of a feature extraction CNN; and

FIG. 11 is a flowchart of blood oxygen saturation calculation.

Reference Numerals: 1. eye mask body; 2. inner eye wear; 3. outer eye wear; 4. ear-loop elastic strap; 5. left EEG electrode; 6. right EEG electrode; 7. LEOG electrode; 8. REOG electrode; 9. left reference electrode; 10. right reference electrode; 11. PPG sensor; 12. hardware circuit; 13. power battery; 14. switch; 15. LED light; 161. hook side; and 162. loop side.

### DETAILED DESCRIPTION OF THE EMBODIMENTS

The technical solutions of the present invention will be clearly and completely described below in conjunction with specific embodiments and drawings of the present invention. Obviously, the described embodiments are only a part, rather than all of the embodiments of the present invention. All other embodiments obtained by those skilled in the art based on the embodiments of the present invention without creative efforts should fall within the protection scope of the present invention.

In the description of the present invention, orientation or position relationships indicated by terms such as “upper”, “lower”, “front”, “rear”, “inside”, “outside”, “left”, and “right” are based on the drawings. These terms are merely intended to facilitate description of the present invention and simplify the description, rather than to indicate or imply that the mentioned device or element must have a specific orientation and be constructed and operated in a specific orientation. Therefore, these terms should not be construed as a limitation to the present invention. Terms such as “first” and “second” are intended to distinguish between similar objects, rather than to indicate specific order or importance. Those of ordinary skill in the art may understand specific meanings of the foregoing terms in the present invention based on a specific situation. In addition, the terms “including” and “having” and any variations thereof are intended to cover non-exclusive inclusions that may include other units not clearly listed or inherent to these products or devices.

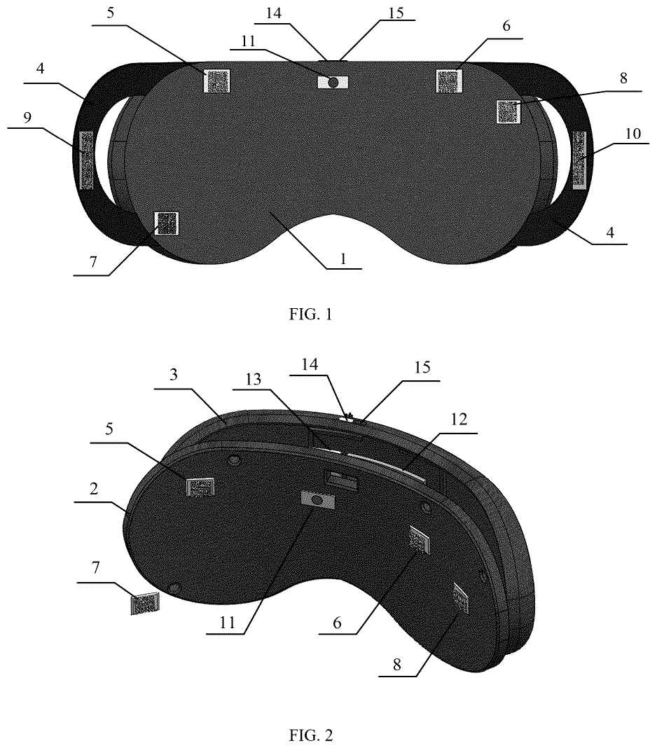

As shown in FIG. 1, Embodiment 1 of the present invention provides a sleep-related breathing disorder monitoring eye mask, mainly including eye mask body 1, a pair of EEG electrodes (electrode 5 and electrode 6), a pair of EOG electrodes (electrode 7 and electrode 8), a pair of reference electrodes (electrode 9 and electrode 10), PPG sensor 11, hardware circuit 12, and power battery 13. The eye mask body 1 includes an eye wear and ear-loop elastic straps 4. The eye wear includes inner eye wear 2 and outer eye wear 3. Both the inner eye wear 2 and the outer eye wear 3 are made of a cotton fabric with good flexibility, comfort, and breathability, or a material such as silk, leather, or elastic fiber. The inner eye wear 2 and the outer eye wear 3 form an integral whole with respective edges fixedly connected. There are two ear-loop elastic straps 4. They are fixedly connected to two sides of the eye mask body 1, respectively, configured to secure the eye mask body 1 to a head, and can be made of a material such as spandex, polyester, or elastic fiber. In other embodiments, the elastic strap can adopt a headband style, and the present invention imposes no limitation on this. It can be understood that the eye mask body formed by the inner eye wear 2, the outer eye wear 3, and the ear-loop elastic straps 4 constitutes a head-worn wearable mechanism to carry the aforementioned electrodes and components.

On an inner side of the eye mask body 1 (i.e., a side contacting a skin), the two EEG electrodes, namely the electrode 5 and the electrode 6, are symmetrically fixed to upper left and right sides of the inner eye wear 2, respectively, and the two EOG electrodes, namely the electrode 7 and the electrode 8, are fixed to a lower left side and an upper right side of the inner eye wear 2, respectively. As shown in FIG. 3, when the eye mask is in a worn state, the EEG electrodes are located precisely at left and right sides of the prefrontal region (Fp1 and Fp2 in standard EEG electrode placement) respectively to acquire EEG potential signals. The EOG electrodes are located approximately 1 cm below the left outer canthus and approximately 1 cm above the right outer canthus. The EOG signal is formed by a potential difference between the two EOG electrodes. The two reference electrodes, namely the left reference electrode 9 and the right reference electrode 10, are fixed to the inner sides of the left and right ear-loop elastic straps 4, respectively. As shown in FIG. 4, when the eye mask is worn, the two reference electrodes respectively contact mastoid processes posterior to junctions of left and right auricles with cheeks to provide stable, low-amplitude reference potentials. Each EEG signal is formed by the potential difference between an EEG electrode (left or right) and its corresponding reference electrode. The aforementioned electrodes can be fixed to the inner eye wear 2 or the ear-loop elastic straps 4 using ointment-like adhesives such as silicone.

It is worth noting that in the present invention, the potential difference between the potential signals acquired by the left and right EEG electrodes located on the forehead and the potential difference between the reference signals acquired by the left and right reference electrodes form two EEG signals. Compared to the occipital region, the area adjacent to the mastoid region is less affected by head movement and free from complex external signal interference (e.g., ECG signals). Compared to the forehead and the tip of the nose, the skin adjacent to the mastoid process is thinner and has lower sweat production, ensuring long-term stability of the electrode-skin interface. Meanwhile, the mastoid process behind the ear is a bony protrusion with a thicker bone structure that can isolate signals from brain activity. Furthermore, the mastoid process is located in the middle and rear of the entire skull, far from the main electrical activity areas of the cerebral cortex, enabling the provision of a reference potential unaffected by brain activity as the reference signal.

Furthermore, in the present invention, the specific electrode placement is designed based on the anatomical features of Asian adults. The human forehead length (from the hairline to the upper edge of the eye) is approximately 6-8 cm, the forehead width (from the hairline on both sides to the inner canthi of the eyes) is approximately 10-12 cm, the intercanthal distance (horizontal distance between the inner canthi of the eyes) is approximately 2.5-3.5 cm, and the eye length (horizontal distance from the outer canthus to the inner canthus) is approximately 2.2-3.0 cm. Based on these measurements, the EEG electrodes (the electrode 5 and the electrode 6) are placed at 4-6 cm left and right from the midline and 0.5-2.5 cm from the upper edge respectively, the LEOG electrode (the electrode 7) is placed at 6-8 cm left and right and 0.5-2.5 cm from the lower edge, and the REOG electrode (the electrode 8) is placed at 4-6 cm from the upper edge.

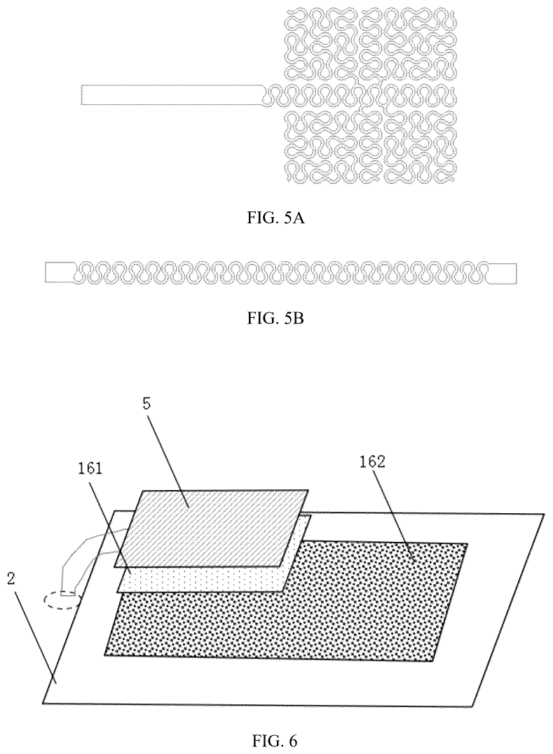

Furthermore, in the present invention, a flexible hook-and-loop fastener is used cooperatively to ensure accurate positioning. The flexible hook-and-loop fastener ensures movable positions of the monitoring electrodes (i.e., the EEG and EOG electrodes), ensuring the set positions are reusable and always within the ideal scope. The hook-and-loop fastener typically includes a loop side and a hook side, achieving adhesion through the cooperation of the loop side and the hook side. As shown in FIG. 6, for example, the monitoring electrode has a size of 1×1 cm². The monitoring electrode is bonded to the hook side 161 of the same shape and size via adhesive backing. The loop side 162 has a size of 2×2 cm². The loop side 162 is bonded to the inner eye wear 2 via the adhesive backing. Thus, during initial wear, the user can adjust the specific position where the monitoring electrode is attached to the loop side 162 through the hook side 161 based on the facial structure characteristics of the user and the manual, eliminating the need for readjustment during subsequent wear.

To ensure good contact between various types of electrodes and the skin, the electrodes (EEG, EOG, and reference electrodes) need to be flexible, able to deform and adapt to different worn states like the fabric of the eye mask. Additionally, the ear-loop elastic straps of the eye mask need to be repeatedly stretched during wear. Therefore, the reference electrodes and connecting wires need sufficient stretchability to avoid breakage during strap stretching, thereby ensuring stable signal acquisition and transmission. As shown in FIG. 5A, these electrodes each can be prepared based on an ultra-thin metal foil with a serpentine pattern and formed via high-precision laser cutting. Compared to rigid electrodes like silver/silver chloride or gold cup electrodes, flexible electrodes offer higher skin conformability and comfort, effectively avoiding poor contact due to uneven skin. As shown in FIG. 5B, the wires between each electrode and the hardware circuit can also adopt serpentine routing to effectively prevent circuit breakage caused by eye mask stretching and deformation. Furthermore, an ultra-thin and stretchable polyimide (PI) film can be used for insulating encapsulation of the serpentine routing via methods like hot melting and hot pressing, ensuring stable and reliable signal transmission.

The PPG sensor 11 mainly includes a light source (e.g., light-emitting diode (LED) light) and a photodetector. The PPG sensor 11 is fixed to the upper middle part of the inner eye wear via a hole cut on the surface of the inner eye wear 2. As shown in FIG. 3, when the eye mask is worn, the PPG sensor 11 is located at the center of the forehead to monitor the PPG signal (referred to as “PPG signal”) of the forehead through the reflected PPG signal. PPG is a non-invasive technique configured to measure changes in blood flow within blood vessels by detecting optical changes in the skin. When light emitted by the light source passes through the skin, blood vessels, and blood, part of the light is reflected and received by the detector. As hemoglobin binds with oxygen, light of different wavelengths is absorbed, thereby affecting the light intensity received by the sensor. In this embodiment, the PPG sensor 11 can specifically be a MAX30102 sensor.

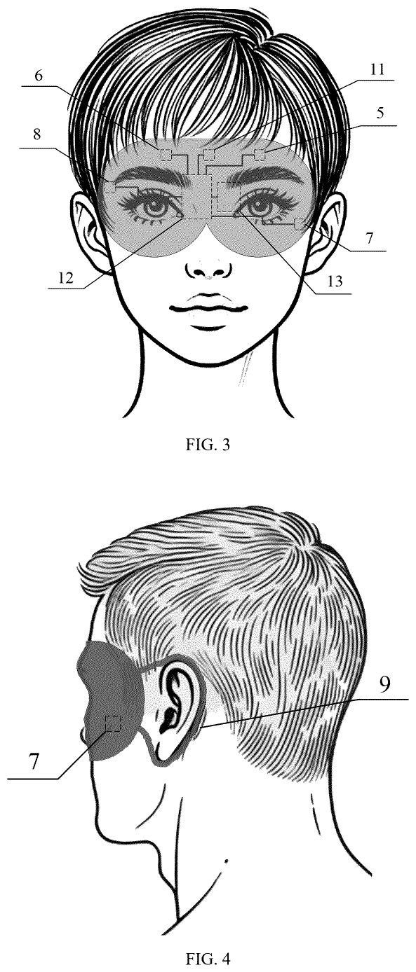

As shown in FIG. 2 and FIG. 3, the hardware circuit 12 and the power battery 13 can be fixed between the inner eye wear 2 and the outer eye wear 3 by adhesion, and are located precisely at the center of the forehead to balance the overall weight when the eye mask is worn. Multiple pin connectors of the hardware circuit are respectively connected to the EEG electrodes, the EOG electrodes, the reference electrodes and the PPG sensor via wires to synchronously acquire EEG, EOG, and PPG signals. The top of the outer eye wear 3 is provided with switch 14 electrically connected to the hardware circuit 12. The switch 14 is configured to control the on/off state of the sleep-related breathing disorder monitoring eye mask.

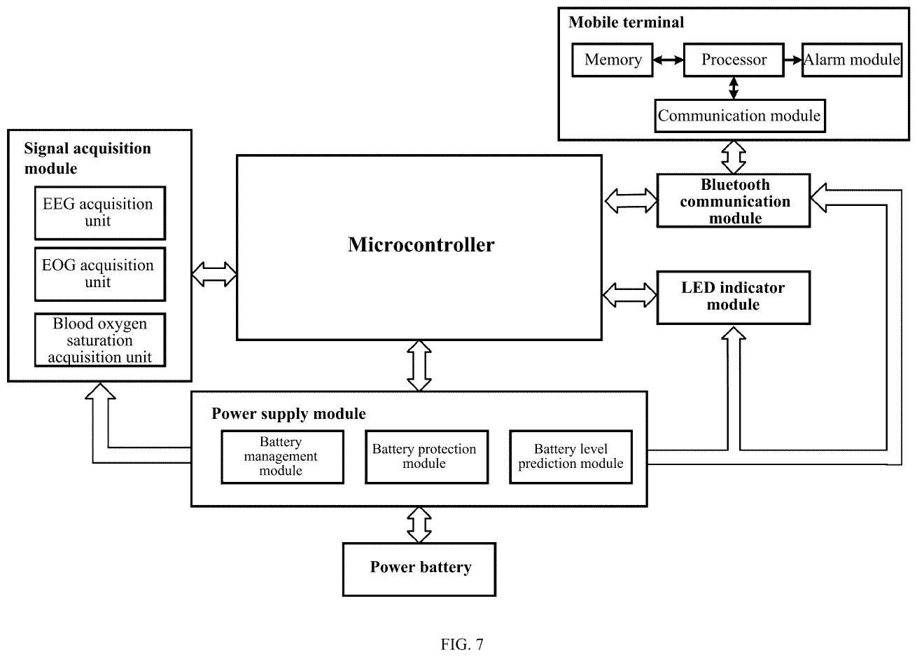

As shown in FIG. 7, the hardware circuit 12 integrates a microcontroller, which is primarily configured for data processing and module function control and can adopt an STM32 controller. The hardware circuit 12 also integrates a signal acquisition module with multiple signal acquisition units. The signal acquisition units each are integrated with an analog amplification circuit and an analog-to-digital converter (ADC) circuit to perform amplification and analog-to-digital conversion of the EEG, EOG, and PPG signals, respectively. For example, the analog amplification circuit in the EEG processing unit first amplifies the minute-amplitude EEG signals. The ADC circuit converts the amplified analog signals into digital signals. The hardware circuit 12 also integrates a Bluetooth communication module and a power management module. The microcontroller transmits the signals after analog-to-digital conversion to a mobile terminal via the Bluetooth communication module. The power management module is connected to the power battery 13, primarily configured to supply power to the microcontroller and various functional modules in the hardware circuit 12. Furthermore, the hardware circuit 12 also includes an LED indicator module for indicating the system's on/off state. The hardware circuit 12 can be a flexible printed circuit (FPC) board made with a PI or polyester film as the substrate, characterized by high reliability, light weight, small thickness, and good bendability.

Embodiment 2 of the present invention provides a mobile terminal. The mobile terminal, together with the sleep-related breathing disorder monitoring eye mask in Embodiment 1, constitutes a sleep-related breathing disorder monitoring system. The mobile terminal includes a processor, and a memory, a communication module and an alarm module that are electrically connected to the processor. The mobile terminal specifically can be an electronic device such as a smartphone, a tablet computer, a smartwatch, a smart bracelet, or a dedicated companion terminal. The alarm module specifically can be implemented via a speaker built into the mobile terminal.

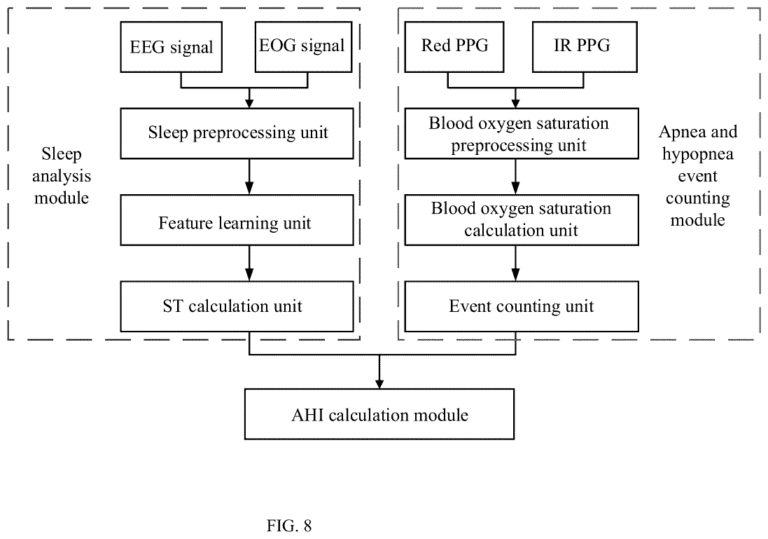

As shown in FIG. 8, the functions of the mobile terminal mainly include automatic sleep staging, apnea and hypopnea event counting, and real-time AHI calculation and updating based on results of sleep staging and apnea and hypopnea event counting. Correspondingly, the processor of the mobile terminal is provided with a sleep analysis module, an apnea and hypopnea event counting module, and an AHI calculation module. The sleep analysis module mainly includes a sleep preprocessing unit, a sleep staging unit, and a ST calculation unit, configured to preprocess EEG and EOG signals, derive a sleep stage prediction result, and calculate the ST, respectively. The apnea and hypopnea event counting module mainly includes a blood oxygen saturation preprocessing unit, a blood oxygen saturation calculation unit, and an event counting unit, configured to preprocess the PPG signal, calculate the blood oxygen saturation, and count the apnea and hypopnea events, respectively. The AHI calculation module is configured to calculate the AHI value in real time based on the sleep staging result from the sleep analysis module and the event counting result from the apnea and hypopnea event counting module.

In a specific embodiment, the acquisition frequency for the EEG and EOG signals in the hardware circuit is set to 100 Hz, with a 30-second epoch for sleep staging. The sleep preprocessing unit of the sleep analysis module applies low-pass filtering to the EEG and EOG signals within a 0-30 Hz range. Additionally, to avoid marginal effects caused by filtering, the length of the input signal for filtering is greater than one epoch (30 s), and after filtering, it is truncated to the length of one epoch. This embodiment adopts a time-lag method, meaning the filtering start time is 10 seconds after data acquisition is complete. After an input signal of 50 seconds is filtered, the filtered signal is vertically concatenated into an EEG signal (array 1) with dimensions (2,3000) and an EOG signal (array 2) with dimensions (1,3000) as the result of sleep preprocessing, and the result of sleep preprocessing is then input into the sleep staging module.

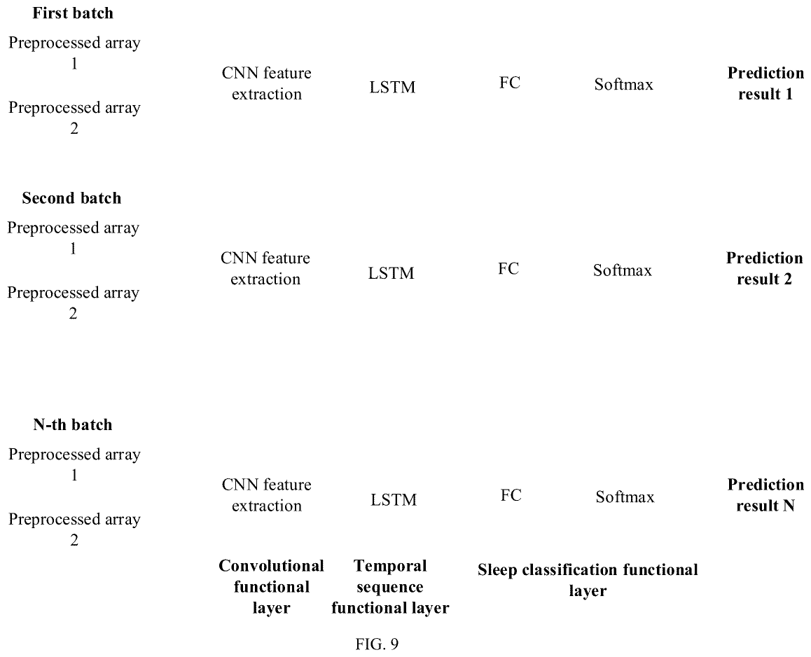

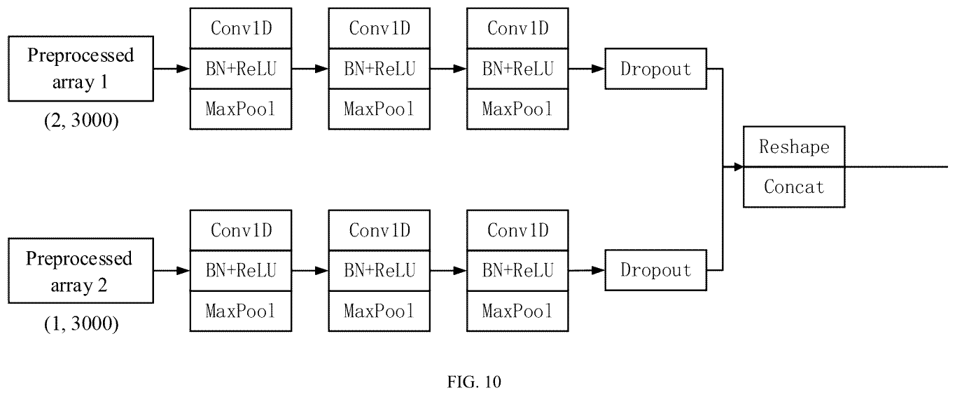

As shown in FIG. 9, the sleep staging unit mainly includes three sequentially connected functional layers: a convolutional functional layer, a temporal sequence functional layer, and a sleep classification functional layer. The convolutional functional layer is shown in FIG. 10, where the array 1 and the array 2 each pass through three consecutive convolutional layers. In each convolutional layer, the array undergoes one-dimensional convolution (Conv1D) to extract high-dimensional features of the array, followed by batch normalization (BN) to transform the feature values into a standard normal distribution with a mean of 0 and variance of 1. The normalized feature array is nonlinearly mapped via a rectified linear unit (ReLU) activation function, and local maxima of the array are selected by a max pooling layer (MaxPool) to reduce the array size, yielding a single-layer convolutional feature array. The single-layer convolutional feature array is then input to the next convolutional layer, repeating the convolution calculation, BN, activation function mapping, and MaxPool operations. The three-layer convolutional feature arrays acquired by processing through the three convolutional layers pass through a dropout layer. A portion of the features are randomly set to 0 to reduce the risk of network overfitting, yielding the output features of the three convolutional layers (i.e., the output features of one branch). The parameters of the convolutional layers for the two branches (array 1 and array 2) are different, resulting in different extracted feature sizes. Therefore, a reshape layer is used to transform the output features of both branches to the same size, followed by concatenation via a Concat layer, resulting in the final convolutional features (CNN features). CNN features from different time periods are sorted chronologically to form a convolutional feature sequence. In the temporal sequence functional layer (LSTM), CNN features at different time periods in the sequence are selected to learn the temporal relationships among the CNN features. The CNN features are transformed and dimensionality-reduced to obtain modified hybrid features. Finally, in the sleep classification functional layer, the modified hybrid features are mapped from high-dimensional features to an output array with dimensions (1,5) via a fully connected (FC) layer. A Softmax layer converts the output array into a probability array based on the dimensions of the output array. Each element in the probability array has a value between 0 and 1, and the sum of all elements is 1. The values in the probability array represent the predicted probabilities for five different sleep stages, including W stage (wakefulness), N1 stage, N2 stage, N3 stage, and REM stage. The sleep stage corresponding to the highest probability is the final prediction result.

The ST calculation unit calculates the wearer's ST based on the sleep staging prediction result: epoch duration (30 s)×total number of sleep stage prediction results (N1+N2+N3+REM).

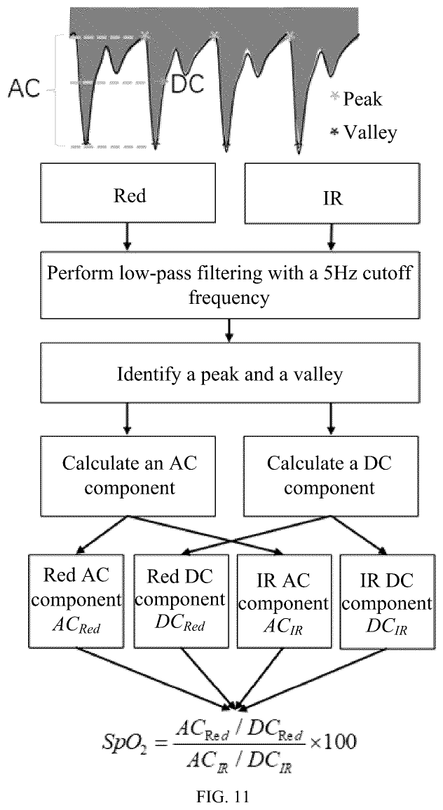

As shown in FIG. 11, red light (Red) and infrared light (IR) emitted by the light source in the PPG sensor 11 are reflected by the skin and received by the detector, forming the PPG signal. For example, the blood oxygen saturation is calculated using a beat-to-beat method. First, the red light and infrared light signals within the PPG signal undergo 5 Hz low-pass filtering in the blood oxygen saturation preprocessing unit. After filtering, the peaks and valleys of the waveform for both signals are identified. The absolute difference between a peak and its corresponding valley within one cardiac cycle is taken as the alternating current (AC) value for that cardiac cycle. The mean value of all low-pass filtered signals between consecutive peaks is taken as the direct current (DC) value for that cardiac cycle.

The blood oxygen saturation calculation unit derives the blood oxygen saturation for the cardiac cycle according to Eq. (1):

$${SpO}_{2} = \frac{( {AC}_{Red} / {DC}_{Red} )}{( {AC}_{IR} / {DC}_{IR} )} \times 100 \qquad ( 1 )$$

where, SpO₂ denotes the blood oxygen saturation; AC_{Red} denotes the AC component of the red light; DC_{Red} denotes the DC component of the red light; AC_{IR} denotes the AC component of the infrared light; and DC_{IR} denotes the DC component of the infrared light.

In the above method, the calculation frequency of the blood oxygen saturation is related to the frequency of cardiac pulsation (HR). To stabilize the sampling rate of the blood oxygen saturation signal, a time window method is used to calculate the blood oxygen saturation value per second, ensuring a sampling rate of 1 Hz for the blood oxygen saturation. First, time window ΔT of specific duration (taking 5 seconds as an example) is set. Within this time window, a newly acquired PPG is stored every second, and the frontmost 1 second of PPG is released. The main peaks and valleys of the PPG are identified within the data of the time window. The average of the absolute differences between each peak and its corresponding valley is taken as the AC value within this time window, and the mean of all values is taken as the DC value for the pulsations within this time window. Then the blood oxygen saturation is calculated according to Eq. (2).

According to The AASM Manual for the Scoring of Sleep and Associated Events: Rules, Terminology and Technical Specifications, a hypopnea event is scored when the blood oxygen saturation decreases by ≥4% from the baseline value. When the wearer experiences an apnea, the respiratory airflow of the wearer nearly stops, and the blood oxygen saturation decreases more rapidly. The difference ΔSpO₂ of the blood oxygen saturation is calculated according to Eq. (2). It can be understood that for the time window method, ΔSpO₂ is the difference between the blood oxygen saturation values of two adjacent seconds. The event counting unit calculates a number of occurrences with the difference exceeding a specified threshold as the number of apnea and hypopnea events (BSN). In this embodiment, the threshold is selected as 4%, which can also be adjusted based on the severity of the monitored sleep apnea and individual differences.

$$Δ {SpO}_{2} = {SpO}_{2} ( t ) - {SpO}_{2} ( t - 1 ) \qquad ( 2 )$$

The AHI calculation module calculates the AHI based on the ST and the number of apnea and hypopnea events (BSN) according to Eq. (3):

$$AHI = \frac{BSN}{ST} \qquad ( 3 )$$

Furthermore, in the present invention, an awakening threshold is set. When the AHI is greater than the awakening threshold, the mobile terminal awakens the wearer via an alarm. In this embodiment, the awakening threshold is set to 15 to awaken wearers with moderate sleep apnea. In other embodiments, modules such as a bone conduction headphone or vibrator can be added to the ear-loop elastic straps 4 of the sleep-related breathing disorder monitoring eye mask. These modules execute corresponding operations to awaken the wearer after receiving relevant instructions from the mobile terminal.

It is worth noting that in the present invention, since each acquired 30-second sleep epoch signal can be used for real-time analysis of ST and BSN, the AHI can be calculated without requiring overnight monitoring, thereby achieving real-time calculation. In fact, AHI changes in real time with sleep time. Therefore, the wearer's AHI changes can be derived through real-time AHI calculation. The wearer's overnight apnea severity can be accurately analyzed based on the dynamic, instantaneously responsive AHI. The awakening threshold can be set according to actual needs to perform timely sleep intervention in severe apnea situations so as to avoid accidents.

Furthermore, the present invention can determine the severity level of OSA based on the AHI and blood oxygen saturation. As shown in Table 1, according to the Primary Care Diagnosis and Treatment Guidelines for OSA in Adults, the severity of OSA in Adults is determined based on the AHI and blood oxygen saturation, and can be divided into three categories: mild, moderate, and severe. Therefore, the severity level OSA in the wearer can be output according to this standard.

TABLE 1
Severity classification of OSA in adults

| Severity | AHI (times/h) | Minimum Blood oxygen saturation (%) |
|---|---|---|
| Mild | 5-15 | 85-90 |
| Moderate | >15-30 | 80-<85 |
| Severe | >30 | <80 |

It can be understood that in other embodiments, the functions implemented by the mobile terminal can also be integrated into the hardware circuit of the sleep-related breathing disorder monitoring eye mask, with relevant data calculation and analysis performed by the microcontroller.

In summary, the present invention designs an integrated head-worn wearable device based on multi-modal signals including EEG, EOG, and blood oxygen saturation. The present invention uses a flexible electronic textile to ensure high wearing comfort. The present invention optimizes electrode selection while meeting sleep diagnosis requirements, solving the problems of EEG signals being interfered by eye movements and excessive common-mode interference caused by insufficient inter-electrode spacing. The present invention places the reference electrodes adjacent to the mastoid processes posterior to the auricles, ensuring the stability of the reference potential. The present invention enables simultaneous wireless transmission and multi-channel data analysis of EEG, EOG, and blood oxygen saturation signals, thereby achieving sleep staging and apnea diagnosis.

To use the sleep-related breathing disorder monitoring system provided in the embodiment of the present invention, first, the power switch 14 is turned on to activate the sleep-related breathing disorder monitoring eye mask. The LED light 15 flashes to indicate that the system is activated, and the mobile terminal establishes inter-device wireless communication. The user wears the smart eye mask. The user can slightly adjust the position of the eye mask by hand to ensure the EEG electrodes (5, 6), the EOG electrodes (7, 8), the reference electrodes (9, 10), and the PPG sensor 11 are in correct positions and in good contact. After the mobile terminal is connected to the sleep-related breathing disorder monitoring eye mask, the LED light maintains constant illumination, and sleep-related breathing disorder monitoring begins. The hardware circuit of the sleep-related breathing disorder monitoring eye mask acquires the EEG, EOG, and blood oxygen saturation signals in real time. These signals are amplified, converted (by the ADC circuit), and wirelessly transmitted to the mobile terminal. The mobile terminal analyzes the EEG and EOG data in real time based on the received signals (the EEG and EOG signals, etc.), performs automatic sleep staging, calculates the blood oxygen saturation, counts sleep apnea and hypopnea events, and calculates the AHI in real time. In case an apnea triggers a warning, the wearer is awakened by an alarm from the sleep-related breathing disorder monitoring eye mask. After the sleep ends, the power switch 14 is turned off. The mobile terminal stops receiving data and no longer displays the analysis results of sleep staging and apnea.

Finally, it should be noted that although the implementations of the present invention are described above with reference to the drawings, the present invention is not limited to the aforementioned specific implementations and application fields, and the above specific implementations are merely illustrative and instructive, rather than restrictive. Those of ordinary skills in the art can make various forms under the inspiration of this specification and without departing from the protection scope of the claims of the present invention, and these forms all fall within the protection scope of the present invention.

1. A sleep-related breathing disorder monitoring method, applied to a head-worn sleep-related breathing disorder monitoring system, and comprising:
acquiring an electroencephalogram (EEG) signal, an electrooculogram (EOG) signal, and a photoplethysmography (PPG) signal;
performing automatic sleep staging and sleep time (ST) calculation based on the EEG signal and the EOG signal;
counting apnea and hypopnea events based on a blood oxygen saturation calculated from the PPG signal; and
calculating an apnea-hypopnea index (AHI) based on a calculated sleep time (ST) and the number of apnea and hypopnea events.

2. The sleep-related breathing disorder monitoring method according to claim 1, further comprising:
preprocessing the PPG signal to obtain a preprocessed PPG signal; and
calculating the blood oxygen saturation based on the preprocessed PPG signal.

3. The sleep-related breathing disorder monitoring method according to claim 1, further comprising:
outputting a severity level of an obstructive sleep apnea (OSA) based on the AHI and the blood oxygen saturation according to a preset rule.

4. The sleep-related breathing disorder monitoring method according to claim 1, further comprising:
issuing a wake-up signal or a wake-up instruction when the AHI is greater than a preset value.

5. The sleep-related breathing disorder monitoring method according to claim 1, wherein
the performing automatic sleep staging and ST calculation comprises:
preprocessing the EEG signal and the EOG signal, and generating an EEG signal and an EOG signal of preset dimensions;
respectively inputting the EEG signal and the EOG signal of the preset dimensions into a sleep staging network based on a preset sleep epoch, and generating a sleep staging prediction result, wherein the sleep staging network is configured to extract a signal feature and a temporal sequence relationship of the signal feature; and the sleep staging prediction result comprises a W stage, an N1 stage, an N2 stage, an N3 stage, and a rapid eye movement (REM) stage; and
calculating the ST based on the sleep staging prediction result, wherein ST=(preset sleep epoch)×(total number of N1 stage, N2 stage, N3 stage, and REM stage in the sleep staging prediction result); and
the counting the apnea and hypopnea events comprises:
calculating a difference between two consecutive blood oxygen saturations, and calculating a number of occurrences with the difference exceeding a preset threshold as the number of apnea and hypopnea events BSN.

6. The sleep-related breathing disorder monitoring method according to claim 5, wherein the sleep staging network comprises:
a convolutional functional layer, configured to: input the EEG signal and the EOG signal of the preset dimensions into two branches, respectively, apply three convolution operations, randomly set a portion of features to 0 through a Dropout layer, and obtain output features from the two branches; transform the output features from the two branches into same dimensions through a Reshape layer, concatenate the output features through a Concat layer, and obtain convolutional features; and sort the convolutional features of different time periods in a chronological order to form a convolutional feature sequence, wherein the convolution operation comprises convolution calculation, batch normalization (BN), application of an activation function, and max pooling (MaxPool) operation;
a temporal sequence functional layer, configured to: select the convolutional features at different time periods in the convolutional feature sequence, learn a temporal relationship among the convolutional features, and generate a modified hybrid feature after transformation and dimensionality reduction; and
a sleep classification functional layer, configured to: pass the modified hybrid feature through a fully connected (FC) layer and a Softmax layer, and output a probability array with a value range of 0-1 to represent prediction probabilities for different sleep stages, wherein a sleep stage corresponding to a maximum probability forms a final sleep staging prediction result.

7. A head-worn sleep-related breathing disorder monitoring system, comprising: a head-worn sleep-related breathing disorder monitoring device and a computer program, wherein
the head-worn sleep-related breathing disorder monitoring device comprises a head-worn wearable mechanism, a pair of EOG electrodes, a pair of EEG electrodes, a pair of reference electrodes, a PPG sensor and a hardware circuit, wherein the pair of EOG electrodes, the pair of EEG electrodes, the pair of reference electrodes, the PPG sensor and the hardware circuit are disposed on the head-worn wearable mechanism;
when the head-worn wearable mechanism is worn, the pair of EOG electrodes are respectively positioned below an outer canthus of a left eye and above an outer canthus of a right eye of a human body to acquire an EOG signal; the pair of EEG electrodes are respectively positioned at left and right sides of a prefrontal region of the human body; the pair of reference electrodes are respectively positioned to contact mastoid processes posterior to junctions of left and right auricles with cheeks of the human body; and the pair of EEG electrodes and the pair of reference electrodes cooperate to acquire an EEG signal;
the PPG sensor is configured to acquire a PPG signal of the human body;
the hardware circuit is configured to preprocess the EOG signal, the EEG signal, and the PPG signal;
one or more computer programs are stored in the hardware circuit and/or an electronic device communicatively connected to the head-worn sleep-related breathing disorder monitoring device; and
the one or more computer programs are executed to implement the sleep-related breathing disorder monitoring method according to claim 1.

8. The head-worn sleep-related breathing disorder monitoring system according to claim 7, wherein the pair of EOG electrodes and the pair of EEG electrodes each are detachably connected to the head-worn wearable mechanism through a flexible hook-and-loop fastener; the hook-and-loop fastener comprises a loop side and a hook side; the loop side is fixed to an inner side of the head-worn wearable mechanism and has an area larger than the hook side; and the pair of EOG electrodes and the pair of EEG electrodes each are bonded to the loop side through the hook side.

9. The head-worn sleep-related breathing disorder monitoring system according to claim 7, wherein when the head-worn wearable mechanism is worn, the pair of EEG electrodes are positioned 4 cm-6 cm left and right respectively from a midline of an inner side of an eye mask and 0.5 cm-2.5 cm from an upper edge of the eye mask, and the pair of EOG electrodes are positioned 6 cm-8 cm left and right respectively from the midline of the inner side of the eye mask, 0.5 cm-2.5 cm from a lower edge of the eye mask, and 4 cm-6 cm from the upper edge of the eye mask.

10. The head-worn sleep-related breathing disorder monitoring system according to claim 7, wherein
the hardware circuit comprises a microcontroller, a signal acquisition module, a first communication module and a power supply module, wherein the signal acquisition module, the first communication module and the power supply module are electrically connected to the microcontroller;
the signal acquisition module is electrically connected to the pair of EOG electrodes, the pair of EEG electrodes and the PPG sensor, and is configured to preprocess the EOG signal, the EEG signal, and the PPG signal of the human body; and
the first communication module is configured to communicatively connect the microcontroller to the electronic device.

11. The head-worn sleep-related breathing disorder monitoring system according to claim 7, wherein the head-worn wearable mechanism is a flexible structure; the hardware circuit is a flexible printed circuit (FPC) board; the pair of EOG electrodes, the pair of EEG electrodes, and the pair of reference electrodes are flexible electrodes; the pair of EOG electrodes and the pair of EEG electrodes each are configured in a stretchable serpentine pattern; and wires for connecting the pair of EOG electrodes, the pair of EEG electrodes, the pair of reference electrodes, the PPG sensor, an intervention component and an internal circuit each is configured in a stretchable serpentine routing.

12. The head-worn sleep-related breathing disorder monitoring system according to claim 11, wherein the head-worn wearable mechanism is an eye mask; the eye mask comprises an eye mask body, wherein the eye mask body is provided with a flexible eye wear and elastic straps; the flexible eye wear comprises an inner eye wear and an outer eye wear connected to each other; the pair of EOG electrodes, the pair of EEG electrodes and the PPG sensor are positioned on the inner eye wear; the pair of reference electrodes are positioned on the elastic straps, respectively; and the hardware circuit is positioned between the inner eye wear and the outer eye wear.

13. An electronic device, comprising: a processor, a memory, and a second communication module, wherein the electronic device is communicatively connected to a head-worn sleep-related breathing disorder monitoring device through the second communication module; the processor is configured to call a computer program stored in the memory to implement the sleep-related breathing disorder monitoring method according to claim 1.

14. The sleep-related breathing disorder monitoring method according to claim 2, wherein
the performing automatic sleep staging and ST calculation comprises:
preprocessing the EEG signal and the EOG signal, and generating an EEG signal and an EOG signal of preset dimensions;
respectively inputting the EEG signal and the EOG signal of the preset dimensions into a sleep staging network based on a preset sleep epoch, and generating a sleep staging prediction result, wherein the sleep staging network is configured to extract a signal feature and a temporal sequence relationship of the signal feature; and the sleep staging prediction result comprises a W stage, an N1 stage, an N2 stage, an N3 stage, and a rapid eye movement (REM) stage; and
calculating the ST based on the sleep staging prediction result, wherein ST=(preset sleep epoch)×(total number of N1 stage, N2 stage, N3 stage, and REM stage in the sleep staging prediction result); and
the counting the apnea and hypopnea events comprises:
calculating a difference between two consecutive blood oxygen saturations, and calculating a number of occurrences with the difference exceeding a preset threshold as the number of apnea and hypopnea events BSN.

15. The sleep-related breathing disorder monitoring method according to claim 3, wherein
the performing automatic sleep staging and ST calculation comprises:
preprocessing the EEG signal and the EOG signal, and generating an EEG signal and an EOG signal of preset dimensions;
respectively inputting the EEG signal and the EOG signal of the preset dimensions into a sleep staging network based on a preset sleep epoch, and generating a sleep staging prediction result, wherein the sleep staging network is configured to extract a signal feature and a temporal sequence relationship of the signal feature; and the sleep staging prediction result comprises a W stage, an N1 stage, an N2 stage, an N3 stage, and a rapid eye movement (REM) stage; and
calculating the ST based on the sleep staging prediction result, wherein ST=(preset sleep epoch)×(total number of N1 stage, N2 stage, N3 stage, and REM stage in the sleep staging prediction result); and
the counting the apnea and hypopnea events comprises:
calculating a difference between two consecutive blood oxygen saturations, and calculating a number of occurrences with the difference exceeding a preset threshold as the number of apnea and hypopnea events BSN.

16. The sleep-related breathing disorder monitoring method according to claim 4, wherein
the performing automatic sleep staging and ST calculation comprises:
preprocessing the EEG signal and the EOG signal, and generating an EEG signal and an EOG signal of preset dimensions;
respectively inputting the EEG signal and the EOG signal of the preset dimensions into a sleep staging network based on a preset sleep epoch, and generating a sleep staging prediction result, wherein the sleep staging network is configured to extract a signal feature and a temporal sequence relationship of the signal feature; and the sleep staging prediction result comprises a W stage, an N1 stage, an N2 stage, an N3 stage, and a rapid eye movement (REM) stage; and
calculating the ST based on the sleep staging prediction result, wherein ST=(preset sleep epoch)×(total number of N1 stage, N2 stage, N3 stage, and REM stage in the sleep staging prediction result); and
the counting the apnea and hypopnea events comprises:
calculating a difference between two consecutive blood oxygen saturations, and calculating a number of occurrences with the difference exceeding a preset threshold as the number of apnea and hypopnea events BSN.

17. The head-worn sleep-related breathing disorder monitoring system according to claim 7, wherein the sleep-related breathing disorder monitoring method further comprises:
preprocessing the PPG signal to obtain a preprocessed PPG signal; and
calculating the blood oxygen saturation based on the preprocessed PPG signal.

18. The head-worn sleep-related breathing disorder monitoring system according to claim 7, wherein the sleep-related breathing disorder monitoring method further comprises:
outputting a severity level of an obstructive sleep apnea (OSA) based on the AHI and the blood oxygen saturation according to a preset rule.

19. The head-worn sleep-related breathing disorder monitoring system according to claim 7, wherein the sleep-related breathing disorder monitoring method further comprises:
issuing a wake-up signal or a wake-up instruction when the AHI is greater than a preset value.

20. The head-worn sleep-related breathing disorder monitoring system according to claim 7, wherein in the sleep-related breathing disorder monitoring method,
the performing automatic sleep staging and ST calculation comprises:
preprocessing the EEG signal and the EOG signal, and generating an EEG signal and an EOG signal of preset dimensions;
respectively inputting the EEG signal and the EOG signal of the preset dimensions into a sleep staging network based on a preset sleep epoch, and generating a sleep staging prediction result, wherein the sleep staging network is configured to extract a signal feature and a temporal sequence relationship of the signal feature; and the sleep staging prediction result comprises a W stage, an N1 stage, an N2 stage, an N3 stage, and a rapid eye movement (REM) stage; and
calculating the ST based on the sleep staging prediction result, wherein ST=(preset sleep epoch)×(total number of N1 stage, N2 stage, N3 stage, and REM stage in the sleep staging prediction result); and
the counting the apnea and hypopnea events comprises:
calculating a difference between two consecutive blood oxygen saturations, and calculating a number of occurrences with the difference exceeding a preset threshold as the number of apnea and hypopnea events BSN.
